(19) United States
(12) Patent Application Publication
(10) Pub. No.: US 2015/0128940 A1
(43) Pub. Date: May 14, 2015

(54) Packaging of a NO/Nitrogen Gaseous Mixture Having A High NO Concentration

(71) Applicant: AIR LIQUIDE SANTE (INTERNATIONAL), Paris (FR)

(72) Inventors: Pierre De Villemeur, Louveciennes (FR); Laurent Lecourt, Cachan (FR)

(21) Appl. No.: 14/400,559

(22) PCT Filed: Apr. 15, 2013
(86) PCT No.: PCT/FR2013/050816

§ 371 Date: Nov. 12, 2014

(30) Foreign Application Priority Data
May 24, 2012 (FR) 1254764

(51) Int. Cl. F17C 5/06; A61M 16/20; A61M 16/12; A61M 16/04; A61M 16/06

(52) U.S. Cl. CPC F17C 5/06; A61M 16/04; A61M 16/06; A61M 16/122; A61M 16/20; A61M 2202/0275; A61M 2209/06
USPC 128/203.12; 141/4; 220/581

(57) ABSTRACT

The invention concerns a method for storing a NO/N₂ mixture in a packaging container (6) comprising an inner volume, characterized in that it holds a NO/N₂ gaseous mixture containing between 1200 and 4500 ppm by volume of NO, the remainder being nitrogen, at a pressure of at least 100 bar in the inner volume of said container (6), the inner volume being less than or equal to 12 litres (water equivalent).

### CROSS REFERENCE TO RELATED APPLICATIONS

This application is a 371 of International Application PCT/FR2013/050816 filed Apr. 15, 2013, which claims priority to French Application No. 1254764 filed May 24, 2012, the entire contents of which are incorporated herein by reference.

### BACKGROUND

The invention relates to a storage and packaging process, and also to a storage system, such as a gas cylinder, that makes it possible to store a mixture of NO and nitrogen having a high concentration, that is to say at least 1200 ppm by volume, preferably at least 1300 ppm by volume.

NO gas is conventionally used at various concentrations ranging from 100 to 800 ppm by volume (hereinafter “ppmv”), the remainder of the gas mixture being nitrogen, for treating pulmonary vasoconstrictions, in particular pulmonary hypertension, in patients who are undergoing, must undergo or have undergone a cardiac surgery operation, or in hypoxic newborns. In this regard, mention may be made of documents EP-A-786264 and EP-1516639.

Cylinders of NO/N₂ that contain NO at the initial concentration typically between 100 and 800 ppmv may adopt various sizes ranging from 2 to 40 liters (equivalent water capacity).

However, the doses of NO administered to patients range from 1 to 40 ppmv and the administration times vary from a few hours to a few days, for example up to 4 days on average, depending on the patient in question and his clinical condition.

The NO must therefore be diluted, typically with air, O₂-enriched air or N₂/O₂ mixtures, prior to being administered to patients in order to decrease the concentration thereof to less than 40 ppmv, that is to say to the desired dose for the patient in question. This dilution is generally carried out in the patient circuit of a ventilator.

Documents WO-A-2005/110441 and U.S. Pat. No. 6,581,599 teach an NO content being between 800 and 10 000 ppm by volume, the remainder being nitrogen. However, these documents indicate that the packaging of NO/nitrogen mixtures in commercially available gas cylinders can only be carried out at maximum contents of 100 ppmv since higher contents of NO are undesirable as they are capable of causing leakages of NO and a spontaneous degradation of the NO into N₂ for high partial pressures of NO.

Yet the crowded state of resuscitation rooms and operating blocks, and the use of compact NO administration and monitoring systems intended to enable easier transport of patients, make it difficult to use NO/N₂ cylinders of large size, that is to say of more than 12 liters (water capacity), typically those of 20 liters. Specifically, such cylinders lead to significant overcrowding in hospital treatment rooms, are difficult to handle for the care staff, pose problems of storage and transport in buildings, etc.

However, reducing the size of the gas cylinders is not sufficient as this leads to a significant loss of autonomy, that is to say that they do not contain a sufficient amount of gas in order to be able to ensure that NO is dispensed for the required treatment time that may spread over several hours, or even several days.

It is therefore necessary to be able to provide small cylinders, that is to say that have an internal volume of less than 12 liters, but that contain a sufficient amount of gas to enable the treatment of a patient for a treatment time of at least 12 to 24 hours without requiring the cylinder to be changed, preferably at least 1 to 4 days, or even longer.

The problem is then to be able to provide NO/N₂ mixtures in a small-sized storage container, that is to say having a capacity (water equivalent) of less than 12 liters, without encountering or while minimizing the aforementioned autonomy problems.

The solution of the invention relates to a process for storing, i.e. packaging, an NO/N₂ mixture in a packaging container comprising an internal volume, characterized in that an NO/N₂ gas mixture containing from 1500 to 4500 ppm by volume (ppmv) of NO and nitrogen for the remainder is stored, at a pressure of at least 100 bar in the internal volume of said container, the internal volume being less than or equal to 12 liters (water equivalent).

### SUMMARY

According to the invention, in order to overcome the reduction in size of the packaging container, typically a gas cylinder, while retaining sufficient autonomy of the container making it suitable to be used for treating patients suffering from pulmonary vasoconstrictions, the content of NO in the container is increased so as to use a smaller amount of NO/N₂ mixture, during the dilution thereof with the oxygen-rich gas dispensed by the medical ventilator supplying the patient circuit to which the container according to the invention is connected.

Specifically, if the concentration of NO in the cylinder is higher, that is to say at least 1200 ppmv, it will be necessary to use a smaller volume of NO/N₂ in order to obtain a same final concentration of NO in the gas supplying the patient, that is to say the target dose set for each patient, namely of the order of 1 to 40 ppmv depending on the case, typically of the order of 5 to 20 ppmv.

Thus, Table 1 below shows the volumes (in ml) of NO/N₂ administered in the inhalation branch of the patient circuit of the ventilator for a volume per minute of 10 l/min of the ventilator (i.e. air/O₂), in order to obtain NO concentrations between 5 and 40 ppmv, as a function of the NO concentration in the cylinder containing the NO/N₂ mixture (100 to 3500 ppmv).

TABLE 1

| | NO concentration in the cylinder (ppmv) | | | | | | | | | | | |
|---|---|---|---|---|---|---|---|---|---|---|---|---|
| NO final dose | 100 | 200 | 225 | 400 | 450 | 800 | 1000 | 1500 | 2000 | 2250 | 2700 | 3500 |
| 5 ppmv | 556 | 256 | 227 | 127 | 112 | 63 | 50 | 33 | 25 | 22 | 19 | 14 |
| 10 ppmv | 1111 | 513 | 455 | 253 | 225 | 126 | 101 | 67 | 50 | 45 | 37 | 29 |
| 20 ppmv | 2222 | 1026 | 909 | 506 | 449 | 252 | 201 | 134 | 100 | 89 | 74 | 57 |
| 40 ppmv | 4444 | 2051 | 1818 | 1013 | 899 | 503 | 402 | 268 | 201 | 178 | 148 | 114 |

As can be seen in Table 1, the volumes of NO administered in order to obtain doses of 5 ppmv are respectively 556 ml for a concentration of 100 ppmv of NO and 14 ml for a concentration of 3500 ppmv.

Likewise, for doses of 40 ppmv, the volumes are 4444 ml for a concentration of 100 ppmv of NO and 114 ml for an NO concentration of 3500 ppmv.

This clearly demonstrates the advantage that there is in using high NO concentrations, that is to say of at least 1200 ppmv, in order to reduce the impact of the volume of NO administered and of the associated dilution on the ventilator parameters (see Table 2) since the higher the concentration, the lower the volume of NO required.

Table 2 illustrates the dilution percentages (%) obtained for NO concentrations from 100 to 3500 ppmv in order to obtain doses ranging from 5 to 40 ppmv, under the same administration conditions as those from Table 1 (i.e. ventilation of 10 l/min).

TABLE 2

| | NO concentration in the cylinder (ppmv) | | | | | |
|---|---|---|---|---|---|---|
| NO final dose | 100 | 200 | 225 | 400 | 450 | 800 |
| 5 ppmv | 5.56% | 2.56% | 2.27% | 1.27% | 1.12% | 0.63% |
| 10 ppmv | 11.11% | 5.13% | 4.55% | 2.53% | 2.25% | 1.26% |
| 20 ppmv | 22.22% | 10.26% | 9.09% | 5.06% | 4.49% | 2.52% |
| 40 ppmv | 44.44% | 20.51% | 18.18% | 10.13% | 8.99% | 5.03% |
| NO final dose | 1000 | 1500 | 2000 | 2250 | 2700 | 3500 |
| 5 ppmv | 0.50% | 0.33% | 0.25% | 0.22% | 0.19% | 0.14% |
| 10 ppmv | 1.01% | 0.67% | 0.50% | 0.45% | 0.37% | 0.29% |
| 20 ppmv | 2.01% | 1.34% | 1.00% | 0.89% | 0.74% | 0.57% |
| 40 ppmv | 4.02% | 2.68% | 2.01% | 1.78% | 1.48% | 1.14% |

As can be seen, for a same treatment set point, the dilution becomes negligible for high NO concentrations, that is to say of at least 1200 ppmv, and hence no longer influences the ventilator parameters and set points. Indeed, the lower the volume of NO, the less it will change the ventilation set points and counteract the beneficial effects of the treatment, in particular the FiO₂.

Thus, Tables 3 to 5 below clearly show the impact of the dilutions on the desired FiO₂ values for initial NO concentrations in the cylinder of 225, 450 and 2000 ppmv.

The FiO₂ is a very important parameter within the context of the illnesses treated by NO (neonatal refractory hypoxemia, ARDS, etc.) given that for these illnesses FiO₂ values of the order of 100 percent may be necessary. This implies that the dilution must be as low as possible in order to retain an FiO₂ value as high as possible and therefore the beneficial effect of the treatment by inhaled NO.

TABLE 3

| | FiO₂ desired (%) | | | | |
|---|---|---|---|---|---|
| | 21 | 23 | 40 | 60 | 100 |
| 225 ppmv NO | FiO₂ obtained (%) | | | | |
| 5 ppmv | 20.52 | 22.48 | 39.09 | 60.00 | 97.73 |
| 10 ppmv | 19.59 | 21.46 | 37.31 | 57.27 | 93.29 |
| 20 ppmv | 17.81 | 19.51 | 33.92 | 52.07 | 84.80 |
| 40 ppmv | 14.57 | 15.96 | 27.75 | 42.60 | 69.39 |

TABLE 4

| | FiO₂ desired (%) | | | | |
|---|---|---|---|---|---|
| | 21 | 23 | 40 | 60 | 100 |
| 450 ppmv NO | FiO₂ obtained (%) | | | | |
| 5 ppmv | 20.76 | 22.74 | 39.55 | 59.33 | 98.88 |
| 10 ppmv | 20.30 | 22.23 | 38.66 | 57.99 | 96.65 |
| 20 ppmv | 19.39 | 21.23 | 36.92 | 55.39 | 92.31 |
| 40 ppmv | 17.64 | 19.32 | 33.61 | 50.41 | 84.01 |

TABLE 5

| | FiO₂ desired (%) | | | | |
|---|---|---|---|---|---|
| | 21 | 23 | 40 | 60 | 100 |
| 2000 ppmv NO | FiO₂ obtained (%) | | | | |
| 5 ppmv | 20.95 | 22.94 | 39.90 | 59.85 | 99.749 |
| 10 ppmv | 20.84 | 22.83 | 39.70 | 59.55 | 99.249 |
| 20 ppmv | 20.63 | 22.60 | 39.30 | 58.95 | 98.254 |
| 40 ppmv | 20.22 | 22.15 | 38.51 | 57.77 | 96.284 |

As can be seen in Tables 3 and 4 relating to low initial contents of NO, namely 225 and 450 ppmv of NO (remainder nitrogen), the decreases of FiO₂ caused by the dilution may represent around 20% to 30% of the desired FiO₂ (i.e. the set point FiO₂), which becomes prejudicial to the treatment by NO since a low FiO₂ value cancels out the positive effects of the NO.

Conversely, as shown in Table 5, using a high concentration of NO (here 2000 ppm of NO in nitrogen) does not modify or else negligibly modifies (i.e. around <1%) the FiO₂ set points thus making it possible to treat patients with O₂ contents of almost 100% over short periods, in order to get through certain extreme situations.

Although an increase in the concentration of NO in the cylinders may appear simple at first glance, it does entail problems linked to the potential toxicity of high contents of NO (>800 ppmv), since any excessive dose, due to a poor dilution for example, may result in a toxic dose being administered to the patient and since, furthermore, the higher the concentration of NO, the greater the risk of forming toxic compounds, such as toxic NO₂ that is formed by oxidation of NO in the presence of oxygen.

In other words, the solution proposed by the invention goes against certain prejudices that exist in the field in question, which consider that the use of NO at high doses, that is to say greater than 1100 ppmv, is dangerous.

Using high concentrations of NO, i.e. more than 1200 ppm by volume (ppmv), makes it possible to reduce the size of the packaging cylinders used and the volumes of NO administered, and therefore to solve the aforementioned overcrowding problems and to make it possible furthermore to adapt the packaging to a daily treatment or treatment per patient in a manner similar to conventional treatments, therefore to enable better monitoring of the treatment received by the patient.

Thus, Tables 6 and 7 below show that the volume of NO needed for a daily treatment (Table 6) or a four-day treatment (Table 7), for an adult patient ventilated at 10 l/min, is, for a target concentration of 20 ppmv:
between 1309 liters for a cylinder containing 225 ppmv and 64 liters for a cylinder containing 4500 ppmv for a daily treatment (1 day);
between 5236 liters for a cylinder containing 225 ppmv and 256 liters for a cylinder containing 4500 ppmv for a treatment of 4 days.

TABLE 6

| | Daily volume (in liters) | | | | | | | | |
|---|---|---|---|---|---|---|---|---|---|
| Final NO | 5 ppmv | 327 | 160 | 80 | 72 | 48 | 32 | 21 | 16 |
| content | 10 ppmv | 655 | 320 | 160 | 145 | 96 | 64 | 41 | 32 |
| | 20 ppmv | 1309 | 640 | 320 | 289 | 193 | 128 | 82 | 64 |

| | | | | | | | | |
|---|---|---|---|---|---|---|---|---|
| Initial NO content in | 225 | 450 | 900 | 1000 | 1500 | 2250 | 3500 | 4500 |
| the cylinder (ppmv) | | | | | | | | |

TABLE 7

| | Volume for 4 days (in liters) | | | | | | | | |
|---|---|---|---|---|---|---|---|---|---|
| Final NO | 5 ppmv | 1309 | 640 | 320 | 289 | 193 | 128 | 82 | 64 |
| content | 10 ppmv | 2618 | 1280 | 640 | 579 | 385 | 256 | 165 | 128 |
| | 20 ppmv | 5236 | 2560 | 1280 | 1158 | 771 | 512 | 329 | 256 |

| | | | | | | | | |
|---|---|---|---|---|---|---|---|---|
| Initial NO content in | 225 | 450 | 900 | 1000 | 1500 | 2250 | 3500 | 4500 |
| the cylinder (ppmv) | | | | | | | | |

Tables 6 and 7 therefore clearly illustrate the difference in packaging size necessary for carrying out a treatment under these conditions, whether it be daily or spread over an average duration of several days, typically 4 days.

Thus, for NO contents of 1200 ppmv and above, small-sized cylinders, that is to say typically of 5 liters (water equivalent) or less could be used, even for treatments of several days, whereas for lower concentrations, larger-sized cylinders, that are therefore bulkier, will be needed, for example 20 liter cylinders for a content of 225 ppmv and a treatment time of 4 days.

By way of indication, Table 8 gives a connection between the characteristics of the cylinders for a treatment by NO of 4 days for 2250 ppmv (according to the present invention) and 225 ppmv (according to the prior art) of NO for filling pressures of 200 bar.

TABLE 8

| Cylinder size/NO | Weight | Height | Diameter |
|---|---|---|---|
| Concentration | (kg) | (cm) | (cm) |
| B2 - 2250 ppm | 2.4 | 360 | 102 |
| B2 - 225 ppm | 24.4 | 960 | 204 |

B2: cylinder with a volume of 2 liters (water equivalent)

B20: cylinder with a volume of 20 liters (water equivalent)

Depending on the case, the process of the invention may comprise one or more of the following technical features:
the container is a gas cylinder having a cylindrical body comprising a neck on which a valve or integrated valve regulator (IVR) is mounted, preferably the valve or IVR are protected by a rigid protective cap or guard, such as that described for example by EP-A-629812.
the container is a gas cylinder having a body formed from one or more composite materials, for example made of fiberglass or of an aluminum alloy.
a container of cylindrical shape having a diameter between 5 and 40 cm and a height between 10 and 80 cm is used.
the NO/N₂ gas mixture contains less than 4000 ppm by volume of NO and nitrogen (N₂) for the remainder.
the NO/N₂ gas mixture contains less than 3500 ppm by volume of NO and nitrogen (N₂) for the remainder.
the NO/N₂ gas mixture contains at least 1300 ppm by volume of NO, preferably from 1500 to 3000 ppm by volume, and nitrogen (N₂) for the remainder.
the NO/N₂ gas mixture contains up to 2500 ppmv of NO.
the NO/N₂ gas mixture is stored at a pressure of from 100 to 500 bar, preferably between 140 and 350 bar.
the NO/N₂ gas mixture is stored at a pressure of at least 200 bar, preferably between 200 and 350 bar.
the internal volume is less than or equal to 11 liters (water equivalent), preferably less than or equal to 5 liters.
the container is a gas cylinder having a body formed from an aluminum alloy comprising aluminum (Al), from 1.8% to 2.6% of copper (Cu), from 1.3% to 2.1% of magnesium (Mg) and from 6.1% to 7.5% of zinc (Zn), preferably an aluminum alloy is used that additionally comprises (% by weight) from 0 to 0.15% of silicon (Si).
the container is a gas cylinder having a body formed from an aluminum alloy comprising (% by weight) from 86.7% to 90.7% of aluminum.
the container is a gas cylinder having a body formed from an aluminum alloy having a density of between 2 and 3.5 g/cm³, preferably between 2.5 and 3 g/cm³, typically of the order of 2.85 g/cm³.
the container is a gas cylinder, the peripheral wall of which has a thickness (E) of less than 30 mm.
the container is a gas cylinder having a body formed from an aluminum alloy.
the container of cylindrical shape comprises, at one end, a base and, at the other end, a neck with an outlet orifice, attached to which is a device for controlling the gas flow and/or for reducing pressure.

The invention also relates to a system for storing an NO/N₂ gas mixture comprising a packaging container having an internal volume of less than or equal to 12 liters (water equivalent), characterized in that the NO/N₂ gas mixture contains from 1500 to 4500 ppm by volume of NO and nitrogen for the remainder, at a pressure of at least 100 bar in the internal volume of said container.

Depending on the case, the storage system of the invention may comprise one or more of the following technical features:
the NO/N₂ gas mixture contains from 1500 to 4000 ppm by volume of NO and nitrogen (N₂) for the remainder.
the NO/N₂ gas mixture contains from 1500 to 3500 ppm by volume of NO and nitrogen (N₂) for the remainder.
the NO/N₂ gas mixture contains from 1500 to 3000 ppm by volume of NO and nitrogen (N₂) for the remainder.
the NO/N₂ gas mixture contains from 1500 to 2500 ppm by volume of NO and nitrogen (N₂) for the remainder.
the NO/N₂ gas mixture is at a pressure of from 100 to 500 bar, preferably between 140 and 350 bar.
the NO/N₂ gas mixture is stored at a pressure of at least 200 bar, preferably between 200 and 350 bar.
the internal volume is less than or equal to 11 liters (water equivalent), preferably less than or equal to 5 liters.

The invention also relates to an installation for dispensing an NO-containing gas to a patient comprising a container that contains an NO/N₂ gas mixture, a ventilator that delivers an oxygen-containing gas, and a patient circuit fluidically connected to the ventilator and to the container containing the NO/N₂ mixture, directly or by means of an NO administration system, characterized in that the container containing the NO/N₂ mixture is one (or more) packaging container(s), such as a gas cylinder, having an internal volume of less than or equal to 12 liters (water equivalent), which internal volume contains an NO/N₂ gas mixture comprising from 1500 to 4500 ppm by volume of NO, and nitrogen for the remainder, at a pressure of at least 100 bar.

### BRIEF DESCRIPTION OF THE DRAWING

The present invention will now be better understood owing to the description given below with reference to the appended FIGURE.

FIG. 1 represents one embodiment of an installation for dispensing NO supplied by a gas packaging system according to the invention

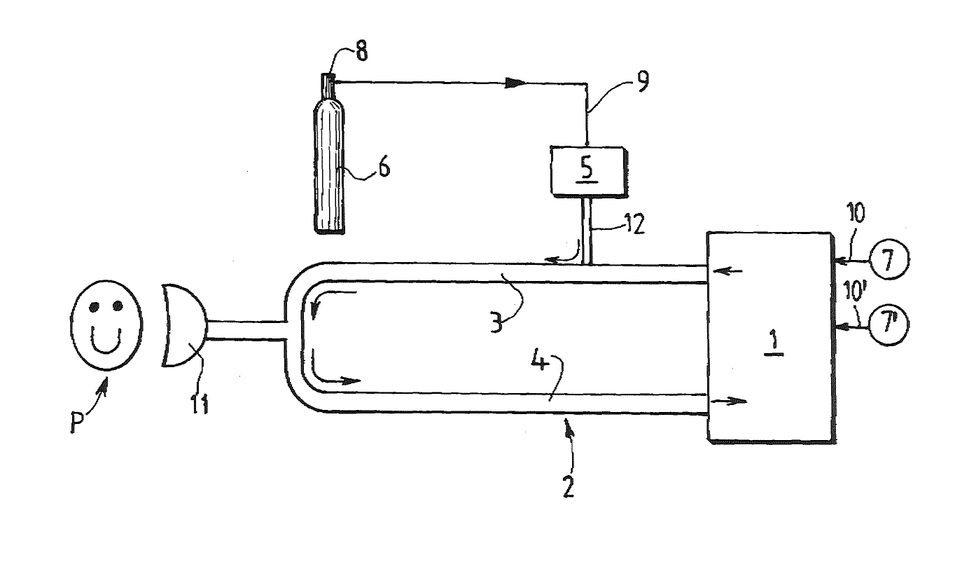

### DESCRIPTION OF PREFERRED EMBODIMENTS

Several cylinders made of an aluminum alloy of metallurgical composition given in the table below are used for storing a gas mixture formed of nitrogen monoxide (NO) and nitrogen (N2) containing a high content of NO, i.e. typically between 1200 and 4500 ppmv.

TABLE 9

| | Proportion (%) by |
|---|---|
| | weight |
| Elements | of the alloy |
| Pb | 0 to 0.003 |
| Fe | 0 to 0.2 |
| Si | 0 to 0.15 |
| Cu | 1.8 to 2.6 |
| Mn | 0 to 0.2 |
| Mg | 1.3 to 2.1 |
| Cr | 0.15 to 0.25 |
| Zn | 6.1 to 7.5 |
| Ti | 0 to 0.05 |
| Zr | 0 to 0.05 |
| Impurities | 0 to 0.15 |
| Al | remainder |
| Density of the alloy | 2.85 g/cm³ |
| | approximately |

The cylinders are equipped with an integrated valve regulator, also referred to as IVR, that makes it possible to control the output of gas from the container 6.

The NO/N₂ gas mixture that comprises an NO content of between 1200 and 4500 ppm, namely here of the order of 1500 ppmv, was introduced under a high pressure into cylinders of B1, B2, B5 and B11 type, the equivalent water capacity of which is, respectively, 1, 2, 5 and 11 liters.

The cylinders thus obtained contained in the end the NO/N₂ mixture at a pressure of the order of 200 bar and at a content of 1500 ppmv.

These cylinders were used for supplying an installation for dispensing NO to patients suffering from pulmonary vasoconstrictions, for example an installation for dispensing NO, one embodiment of which is shown schematically in the appended FIGURE.

This installation comprises a ventilator 1 comprising a respiratory circuit or patient circuit 2 with two branches, that is to say with an inhalation branch 3 and an exhalation branch 4.

The inhalation branch 3 is designed to convey respiratory gas from the ventilator 1 to the patient P, whereas the exhalation branch 4 is designed to convey the gas exhaled by the patient P to the ventilator 1.

At the patient P, the gas is administered by means of a patient interface 11, for example a respiratory mask or a tracheal cannula or catheter.

The ventilator 1 is supplied, via several delivery lines 10, 10′, with air (O₂ content of 21% by volume) originating from an air source 7 and with oxygen resulting from an oxygen source 7′, such as gas cylinders or lines transporting, respectively, medical air and oxygen originating from an oxygen production unit, such as a pressure swing (PSA) unit, or from an oxygen storage unit, such as a buffer or storage tank.

The air is enriched in oxygen in the ventilator 1 and the oxygen-rich gas thus obtained is delivered by the ventilator 1 into the inhalation branch 3 of the patient circuit 2.

Furthermore, a device 5 for dispensing NO is fluidically connected to said inhalation branch 3 of the patient circuit 2 in order to deliver therein, via a feed line 12, an NO/N₂ mixture containing a high concentration of NO, that is to say at least 1200 ppm by volume, according to the present invention.

The device 5 for dispensing NO is itself supplied with an NO/N₂ mixture, via a gas feed line 9, by a container 6 containing an NO/nitrogen mixture, such as an aluminum gas cylinder (cf. Table 9 above) and equipped with a valve or integrated valve regulator 8, preferably protected by a guard for protection against impacts.

The device 5 for dispensing NO makes it possible in particular to control the amount of NO/N₂ released into the inhalation branch 3, and also the method of releasing this mixture, that is to say continuously or in a pulsed manner, for example only during the inhalation phases of the patient P.

Therefore, in the inhalation branch 3, a dilution of the NO/N₂ mixture with the oxygen-rich gas distributed by the ventilator 1 takes place. The dilution depends on the content of the initial NO/N₂ mixture and also on the concentration of gas to be administered to the patient.

The NO/N₂/O₂ mixture obtained after dilution makes it possible to treat pulmonary vasoconstrictions, especially pulmonary hypertension, in a patient that is undergoing, must undergo or has undergone a cardiac surgery operation, or in a hypoxic newborn.

It will be understood that many additional changes in the details, materials, steps and arrangement of parts, which have been described herein in order to explain the nature of the invention, may be made by those skilled in the art within the principle and scope of the invention as expressed in the appended claims. Thus, the present invention is not intended to be limited to the specific embodiments in the examples given above.

1-15. (canceled)

16. A process for storing an NO/N₂ mixture in a packaging container (6) comprising an internal volume, comprising the step of storing a NO/N₂ gas mixture containing between 1200 and 4500 ppm by volume of NO and nitrogen for the remainder at a pressure of at least 100 bar in the internal volume of said container (6), the internal volume being less than or equal to 12 liters (water equivalent).

17. The process of claim 16, wherein the container (6) is a gas cylinder having a cylindrical body comprising a neck to which a valve or valve regulator (8) is fixed.

18. The process of claim 17, wherein the container (6) of cylindrical shape has a diameter between 5 and 40 cm and a height between 10 and 80 cm.

19. The process of claim 16, wherein the NO/N₂ gas mixture contains from 1500 to 4000 ppm by volume.

20. The process of claim 16, wherein the NO/N₂ gas mixture contains from 1500 to 3000 ppm by volume NO, and nitrogen (N₂) for the remainder.

21. The process of claim 16, wherein the NO/N₂ gas mixture contains 1200 to 3500 ppmv of NO.

22. The process of claim 16, wherein the NO/N₂ gas mixture is stored at a pressure of from 140 to 350 bar.

23. The process of claim 16, wherein the NO/N₂ gas mixture is stored at a pressure of 200 to 350 bar.

24. The process of claim 16, wherein the internal volume of the container (6) is less than or equal to 6 liters (water equivalent).

25. A system for storing an NO/N₂ gas mixture comprising a packaging container (6) having an internal volume of less than or equal to 12 liters (water equivalent), wherein the NO/N₂ gas mixture contains from 1200 to 4500 ppm by volume of NO and nitrogen for the remainder, at a pressure of at least 100 bar in the internal volume of said container (6).

26. The storage system of claim 25, wherein the NO/N₂ gas mixture contains from 1500 to 4000 ppm by volume of NO and nitrogen (N₂) for the remainder.

27. The storage system of claim 25, wherein the NO/N₂ gas mixture is at a pressure of from 100 to 500 bar.

28. The storage system of claim 25, wherein the container (6) is equipped with a valve or an integrated valve regulator (8).

29. The storage system of claim 25, wherein the internal volume of the container (6) is less than or equal to 5 liters (water equivalent).

30. An installation for dispensing an NO-containing gas to a patient comprising:
A container (6) that contains an NO/N₂ mixture,
a ventilator (1) that delivers an oxygen-containing gas, and
a patient circuit (2) fluidically connected to the ventilator (1) and to the container (6) containing the NO/N₂ mixture,
wherein that the container (6) containing the NO/N₂ mixture is one (or more) packaging container(s) (6), having an internal volume of less than or equal to 12 liters (water equivalent), which internal volume contains an NO/N₂ gas mixture comprising from 1200 to 4500 ppm by volume of NO, and nitrogen for the remainder, at a pressure of at least 100 bar.
